(19) United States
(12) Patent Application Publication
(10) Pub. No.: US 2021/0285967 A1
(43) Pub. Date: Sep. 16, 2021

(54) A METHOD FOR DIFFERENTIALLY DIAGNOSING IN VITRO A BIPOLAR DISORDER AND A MAJOR DEPRESSIVE DISORDER

(71) Applicants: CENTRE NATIONAL DE LA RECHERCHE SCIENTIFIQUE (CNRS), Paris (FR); UNIVERSITÉ D'AIX-MARSEILLE (AMU), Marseille (FR); UNIVERSITÉ CÔTE D'AZUR, Nice (FR); INSTITUT NATIONAL DE LA SANTÉ ET DE LA RECHERCHE MÉDICALE, Paris (FR); ASSISTANCE PUBLIQUE - HÔPITAUX DE MARSEILLE (AP-HM), Marseille (FR); SORBONNE UNIVERSITE, Paris (FR); UNIVERSITE DE MONTPELLIER, Montpellier (FR); CENTRE HOSPITALIER UNIVERSITAIRE DE MONTPELLIER, Montpellier (FR)

(72) Inventors: Wafa Bel Haj Ali, Paris (FR); El Chérif Ibrahim, La Motte d'Aigues (FR); Raoul Belzeaux, Marseille (FR); Lionel Fillatre, Valbonne (FR); Nicolas Glaichenhaus, Nice (FR); Philippe Courtet, Montpellier (FR); Emanuela Martinuzzi, Biot (FR); Susana Do Carmo Pinto Barbosa, Nice (FR)

(73) Assignees: CENTRE NATIONAL DE LA RECHERCHE SCIENTIFIQUE (CNRS), Paris (FR); INSTITUT NATIONAL DE LA SANTÉ ET DE LA RECHERCHE MÉDICALE, Paris (FR); UNIVERSITÉ D'AIX-MARSEILLE (AMU), Marseille (FR); UNIVERSITÉ CÔTE D'AZUR, Nice (FR); ASSISTANCE PUBLIQUE - HÔPITAUX DE MARSEILLE (AP-HM), Marseille (FR); SORBONNE UNIVERSITE, Paris (FR); UNIVERSITE DE MONTPELLIER, Montpellier (FR); CENTRE HOSPITALIER UNIVERSITAIRE DE MONTPELLIER, Montpellier (FR)

(21) Appl. No.: 17/282,452

(22) PCT Filed: Oct. 4, 2019
(86) PCT No.: PCT/EP2019/076977

§ 371 Date: Apr. 2, 2021

(30) Foreign Application Priority Data
Oct. 4, 2018 (EP) 18198714.0

(51) Int. Cl. G01N 33/68; A61B 5/16

(52) U.S. Cl. CPC G01N 33/6893; G01N 2800/304; G01N 2333/52; A61B 5/165

(57) ABSTRACT

The invention relates to an in vitro or ex vivo method for differentially diagnosing a bipolar disorder and a major depressive disorder in a human patient in a need thereof presenting depressive symptoms, comprising the following steps: providing a biological sample from said patient; determining, from said biological sample, the abundance of at least one of the following cytokines TNF-α, IFN-γ, IL-6, IL-10, IL-12p40, IL-15, IL-16, IL-17A and IL-27; and diagnosing a bipolar disorder or a major depressive disorder from the determination of the abundance of the at least one of the following cytokines TNF-α, IFN-γ, IL-6, IL-10, IL-12p40, IL-15, IL-16, IL-17A and IL-27.

### FIELD OF THE INVENTION

The invention relates to an in vitro or ex vivo method for differentially diagnosing a bipolar disorder and a major depressive disorder in a human patient in a need thereof, presenting depressive symptoms. It also relates to the use of cytokines as biomarkers for differential diagnosis of these disorders.

### BACKGROUND OF THE INVENTION

Both the major depressive disorder (MDD) and the bipolar disorder (BD) are characterized by mood changes and are therefore referred to as affective disorders. Major depressive disorder is characterized by recurrent episodes of low mood and energy levels. Bipolar disorder is characterized by recurrent and alternating episodes of mood and energy-level disturbances, which are increased on some occasions, for example on mania or hypomania, and decreased on others, for example, on depression. In both major depressive disorder and bipolar disorder, changes in mood are often separated by periods of normal mood, known as euthymia.

The major depressive disorder affects more women than men and its overall lifetime prevalence is 16%. In contrast, the bipolar disorder affects men and women equally, is associated with an earlier age of onset compared to the major depressive disorder, and its prevalence is 4-5-fold lower.

Although mania and hypomania are the most recognizable characteristics of the bipolar disorder, depression is its most frequent clinical presentation. Therefore, the patients suffering from a bipolar disorder are much more likely to present to clinicians when they are depressed, especially in outpatient settings. Unfortunately, the clinical presentation of a patient with bipolar disorder when depressed may not differ from that of a patient suffering from a major depressive disorder. This may explain why almost 40% of bipolar disorder patients are initially misdiagnosed with major depressive disorder and why the average delay for patients suffering from a bipolar disorder to be correctly diagnosed is of approximately 7.5 years.

As a consequence of an initial incorrect diagnosis, bipolar disorder patients are often inappropriately treated with antidepressants alone, which may aggravate the course of the illness and worsen the outcome.

Two self-rated screening instruments, the Mood Disorder Questionnaire (MDQ) and The Hypomania/Mania Symptom Checklist (HCL-32), have been designed to assist clinicians in identifying bipolar disorder patients among those who present with depression. The MDQ has a sensitivity of 0.73 and a specificity of 0.90 indicating that it can correctly identify almost three-quarters of patients with bipolar disorder and will screen out bipolar disorder in 9 of 10 patients without the condition. The HCL-32 has a higher sensitivity of 0.8 but a much lower specificity of 0.51. Despite the usefulness of MDQ and HCL-32 screening instruments, they are unfortunately used by a very limited number of general practitioners in primary care where the majority of care for depression is delivered.

With this objective in mind, several authors have searched for neuroimaging, urine or blood biomarkers that could discriminate major depressive disorder and bipolar disorder patients when they were either in a euthymic, or a depressive state. For example, several authors have used noninvasive structural Magnetic Resonance Imaging (MRI) imaging to identify significant differences in brain morphology between bipolar disorder and major depressive disorder patients. While this approach gave promising results, these results were not adjusted for covariates such as age, gender, and past or current treatments, that depending on the study, identified different regions of the brain as determinant for a differential diagnosis. A result that could possibly be explained by methodological differences or by the inclusion of medicated patients in some studies and of non-medicated patients in others.

Other authors have taken advantage of “-omic” technologies to identify blood or urine biomarkers that were present at different levels in major depressive disorder and bipolar disorder patient body fluids. For example, in a recent exploratory study, a combined gas chromatography-mass spectrometry (GC-MS)-based and nuclear magnetic resonance (NMR) spectroscopic-based metabolomic approach allowed for the identification of six urinary metabolite biomarkers: formate, 2,3-dihydroxybutanoic acid, 2,4-dihydroxypyrimidine, phenylalanine, and (3-alanine; that were present at different levels in the urine of major depressive disorder and bipolar disorder patients. This panel of metabolites discriminated major depressive disorder and bipolar disorder patients with an accuracy of 89.6%. Despite this promising result, and as acknowledged by the authors themselves, there are several notable limitations to this study. First, all subjects were of a particular Chinese ethnicity and were recruited from the same hospital which limits the applicability of the findings. Secondly, the sample size of bipolar disorder subjects was relatively small. Thirdly, results were not adjusted for important confounding variables such as sex, age, body mass index and medication. Then, last but not least, it was not clear whether urine samples were collected from patients when they were depressed.

### SUMMARY OF THE INVENTION

Accordingly, a need exists for developing and validating objective laboratory-based tests enabling differential diagnosis between major depressive disorder and bipolar disorder.

In accordance with a first aspect, the invention concerns an in vitro or ex vivo method for differentially diagnosing a bipolar disorder and a major depressive disorder in a human patient in a need thereof presenting depressive symptoms, comprising the following steps:

determining, from a biological sample, the abundance of at least one of the following cytokines TNF-α, IFN-γ, IL-6, IL-10, IL-12p40, IL-15, IL-16, IL-17A and IL-27; and diagnosing a bipolar disorder or a major depressive disorder from the determination of the abundance of the at least one of the following cytokines TNF-α, IFN-γ, IL-6, IL-10, IL-12p40, IL-15, IL-16, IL-17A and IL-27.

Preferably, —the method comprises the steps of: determining, from said biological sample, the abundance of at least two or at least three of the following cytokines TNF-α, IFN-γ, IL-6, IL-10, IL-12p40, IL-15, IL-16, IL-17A and IL-27; and diagnosing a bipolar disorder or a major depressive disorder from the determination of the abundance of the at least two or at least three of the following cytokines TNF-α, IFN-γ, IL-6, IL-10, IL-12p40, IL-15, IL-16, IL-17A and IL-27; —the diagnosing is achieved using the determined abundance of at least one, at least two, at least three, at least four, or at least five of the following cytokines: IL-17A, TNF-α, IL-10, IL-15, IL-27; and at least one, at least two, at least three, at least four, at least five or at least six of the following cytokines IL-10, IL-16, IL-12p40, IFN-γ, IL-6 and TNF-α; —the method comprises the steps of: determining, from said biological sample, the abundance of at least one, at least two or at least three of the following cytokines IL-17A, TNF-α and IL-10; and diagnosing a bipolar disorder or a major depressive disorder from the determination of the abundance of at least one, at least two or at least three of the following cytokines IL-17A, TNF-α and IL-10; —the diagnosis is achieved using at least the determined abundance of IL-10 and/or TNF-α; —the diagnosis is achieved using at least the determined abundance of IL-10 and TNF-α; —the diagnosis is achieved using the determined abundance of one or more additional cytokines including IL-8; —the diagnosis step excludes taking into account the abundance of the following cytokines CCL2, CCL3, CCL4, CCL11, CCL13, CCL17, CCL20, CCL22, CCL26, CXCL10, IL-1-a, IL-1-β, IL-2, IL-4, IL-5, IL-7, IL-8, IL-12p70, IL-13, IL-15, IL-21, IL-22, IL-23, IL-31, and TNF-β into the provided biological sample; —the biological sample is selected from the group consisting of blood, biopsy tissue, blood serum, blood plasma, stool, sputum, cerebrospinal fluid, or supernatant from cell lysate; —the biological sample is selected from the group consisting of blood, blood plasma or blood serum; —the method further comprises the step of determining, from a biological sample, the abundance of at least one, at least two or at least three of the following cytokines TNF-α, IFN-γ, IL-6, IL-10, IL-12p40, IL-15, IL-16, IL-17A and IL-27 and comparing the abundance level of said at least one, at least two or at least three cytokines with reference values; —the method further comprises the step of determining a probability p that a patient is bipolar; —the probability p is calculated using an equation that is of the type:

$$p = \frac{1}{1 + {e}^{X}}$$

wherein X comprises one or an addition of two or more terms βᵢSRᵢ, wherein the variable SRᵢ is equal to the serum concentration of one of the cytokines TNF-α, IFN-γ, IL-6, IL-10, IL-12p40, IL-15, IL-16, IL-17A and IL-27, and value βᵢ is a reference value identified for said cytokine; —the probability p is calculated using an equation that is of the type:

$$p = \frac{1}{1 + {e}^{X}}$$

wherein X comprises one or an addition of two or more terms, including at least one of the following terms β₆F, β₇G, and β₈H, wherein the variable “F” is equal to IL-17A serum concentration; variable “G” is equal to TNF-α serum concentration; and variable “H” is equal to IL-10 serum concentration; —the IL-17A, TNF-α and/or IL-10 serum concentration are expressed in pg/ml; —β₆ is comprised between 0.281 and 0.343 preferentially equal to approximately 0.312, more preferentially equal to 0.312; β₇ is comprised between 0.112 and 0.136, preferentially equal to approximately 0.124, more preferentially equal to 0.124; and β₈ is comprised between 0.171 and 0.209 preferentially equal to approximately 0.190, more preferentially equal to 0.190; and—the probability is calculated taking into account that the patient has been treated using benzodiazepine, antidepressants, neuroleptics or atypical antipsychotics, and/or any other antipsychotics, or not.

In accordance with a second aspect, the invention concerns a use of at least one, at least two or at least three of the following cytokines TNF-α, IFN-γ, IL-6, IL-10, IL-12p40, IL-15, IL-16, IL-17A and IL-27 as biomarkers for differential diagnosis of a bipolar disorder and a major depressive disorder.

### BRIEF DESCRIPTION OF THE DRAWINGS

Other features and aspects of the present invention will be apparent from the following description and the accompanying drawings, in which:

FIG. 1 shows a table detailing the patient clinical characteristic that were considered in the Example 1 and, in particular, the total number of patients, numbers of males and females, proportion of males, age, body mass index, tobacco consumption, HDRS-17 score, previous or ongoing treatments with antidepressants, antipsychotics, benzodiazepine and lithium, are shown for all MDE patients as well as for bipolar disorder (BD) and major depressive disorder (MDD) patients;

FIG. 2 shows a table with the serum protein concentration descriptive statistics of the patients listed in the table of FIG. 1. In this table, the LLOD (in pg/ml), proportion of samples in which protein levels were <LLOD, the minimum, maximum, median and mean concentrations (in pg/ml), and standard error of the mean (SEM) are indicated;

FIG. 3 shows a table comprising the results of the univariate analysis of serum protein levels in BD and MDD patients. For each serum protein, mean concentrations±SD in BD and MDD patients are indicated. Effect sizes, p-values and False Discovery Rates (FDRs) are shown;

FIG. 4 shows the variables associated with increased odds of belonging to the BD diagnosis category. The data show the list of variables that have been included in the regularized regression logistic model, the mean (±SD) weighted coefficients. All variables listed in the left column were included in Model 1. Only the variables which have been selected more than 80% of the time in Model 1 were included in Model 2. Alpha and lambda hyper-parameters for Model 1 were 0.167 and 0.268 respectively. Alpha and lambda hyper-parameters for Model 2 were 0.10185 and 0.1023 respectively;

FIG. 5 shows the penalized logistic regression Odd Ratios (ORs) and Variable Inclusion Probabilities (VIPs). VIP, mean OR and percentile bootstrap 95% confidence interval (CI) are indicated for each variable. The VIP was computed as the percentage of the bootstrap resamples in which each variable was selected by the Elastic Net multivariable classification method. In this figure, VIP >0.8 are outlined in bold;

FIG. 6 illustrates the distribution of predicted probabilities for individuals to be classified as belonging to the BD category. The data show the distribution of predicted probabilities for individuals clinically diagnosed with MDD (dark grey) and BD (light grey); and

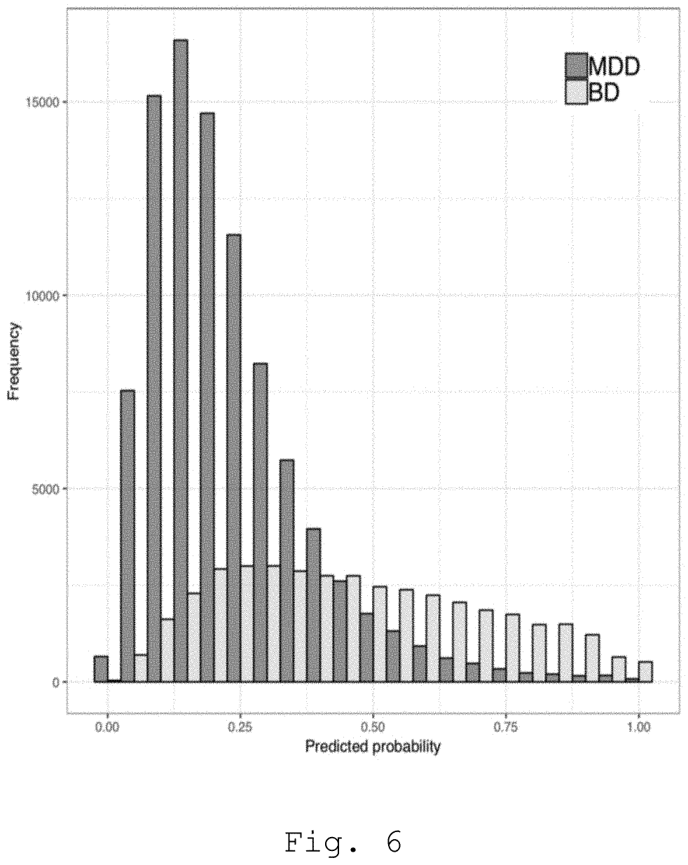

FIG. 7 shows a table detailing the patient clinical characteristic that were considered in the Example 2 and, in particular, the total number of patients, age, body mass index, tobacco consumption, previous or ongoing treatments with antidepressants, antipsychotics, benzodiazepine and lithium, are shown for all MDE patients as well as for bipolar disorder (BD) and major depressive disorder (MDD) patients;

FIG. 8 shows the penalized logistic regression Odd Ratios (ORs) and Variable Inclusion Probabilities (VIPs), for the patients considered in Example 2. VIP, mean OR and percentile bootstrap 95% confidence interval (CI) are indicated for each variable; and

FIG. 9 is a table that indicates the specificity, sensitivity, negative and positive predictive value, the AUROC and optimal threshold that characterizes the results obtained in Example 2.

### DETAILED DESCRIPTION OF THE INVENTION

The invention concerns a method for differentially diagnosing a bipolar disorder and a major depressive disorder in a human patient in a need thereof presenting depressive symptoms. More particularly, the invention concerns an in vitro or ex vivo method for differentially diagnosing a bipolar disorder and a major depressive disorder in a human patient in a need thereof, presenting depressive symptoms, the method comprising a step of determining, from a biological sample of the patient, the abundance of at least one of the following cytokines TNF-α, IFN-γ, IL-6, IL-10, IL-12p40, IL-15, IL-16, IL-17A and IL-27.

Preferably, the invention concerns an in vitro or ex vivo method for differentially diagnosing a bipolar disorder and a major depressive disorder in a human patient in a need thereof presenting depressive symptoms, comprising the following steps: determining, from a biological sample, the abundance of at least one, at least two, at least three, at least four, at least five, at least six, at least seven, at least eight, or at least nine of the following cytokines TNF-α, IFN-γ, IL-6, IL-10, IL-12p40, IL-15, IL-16, IL-17A and IL-27; and diagnosing a bipolar disorder or a major depressive disorder from the determination of the abundance of the at least one, at least two, at least three, at least four, at least five, at least six, at least seven, at least eight or at least nine of the following cytokines TNF-α, IFN-γ, IL-6, IL-10, IL-12p40, IL-15, IL-16, IL-17A and IL-27.

The major depressive disorder and the bipolar disorder are characterized by mood changes and are therefore referred to as affective disorders. The major depressive disorder (MDD) is characterized by recurrent episodes of low mood and energy levels. The bipolar disorder (BP) is characterized by recurrent and alternating episodes of mood and energy-level disturbances, which are increased on some occasions, for example on mania or hypomania, and decreased on others, for example, on depression. In both major depressive disorder and bipolar disorder, changes in mood are often separated by periods of normal mood, known as euthymia.

According to the invention, the patient is a human which is presenting depressive symptoms. It is to be noted that other diagnosing method may be handled, in particular, upfront the diagnosing method according to the invention in order if a patient is mentally healthy or not.

The invention comprises a first step according to which a biological sample of the patient is provided. The biological sample is preferably selected from the group consisting of blood, biopsy tissue, blood serum, blood plasma, stool, sputum, cerebrospinal fluid, or supernatant from cell lysate. More preferably, it is selected from the group consisting of blood, blood plasma or blood serum.

The invention comprises a second step according to which it is determined, from the collected biological sample, the abundance of at least one of the following cytokines biomarkers TNF-α, IFN-γ, IL-6, IL-10, IL-12p40, IL-15, IL-16, IL-17A and IL-27. Preferably, it is determined, from said biological sample, the abundance of at least two of the following cytokines TNF-α, IFN-γ, IL-6, IL-10, IL-12p40, IL-15, IL-16, IL-17A and IL-27. More preferably, it is determined, from said biological sample, the abundance of at least three of the following cytokines TNF-α, IFN-y, IL-6, IL-10, IL-12p40, IL-15, IL-16, IL-17A and IL-27.

According to a mode for carrying out the invention, the in vitro or ex vivo method for differentially diagnosing a bipolar disorder from a major depressive disorder in a human patient in a need thereof presenting depressive symptoms, comprises the following steps: determining, from a biological sample, the abundance of at least one, at least two or at least three of the following cytokines IL-17A, TNF-α and IL-10; and diagnosing a bipolar disorder or a major depressive disorder from the determination of the abundance of the at least one, at least two or at least three of the following cytokines IL-17A, TNF-α and IL-10.

More particularly, the diagnosis is achieved using at least the determined abundance of IL-10 and/or TNF-α. For example, the diagnosis is achieved using at least the determined abundance of IL-10 and TNF-α.

Indeed, as it will be apparent from the examples, two different cohorts have been analyzed. For the first cohort, the five first relevant biomarkers allowing to discriminate MDD from BP that were identified are IL-17A, TNF-α, IL-10, IL-15 and IL-27 and, for the second cohort, the relevant biomarkers are IL-10, IL-16, IL-12p40, IFN-y, IL-6 and TNF-α. It is noted that, as part of these cytokines, IL-10 and TNF-α have been identified as relevant biomarkers for both cohorts.

In one embodiment, the diagnosing is achieved using the determined abundance of at least one, at least two, at least three, at least four, or at least five of the following cytokines: IL-17A, TNF-α, IL-10, IL-15, IL-27.

In one embodiment, the diagnosing is achieved using the determined abundance of least one, at least two, at least three, at least four, at least five or at least six of the following cytokines: IL-10, IL-16, IL-12p40, IFN-γ, IL-6 and TNF-α.

In another embodiment, the diagnosing is achieved using the determined abundance of
at least one, at least two, at least three, at least four, or at least five of the following cytokines: IL-17A, TNF-α, IL-10, IL-15, IL-27; and
at least one, at least two, at least three, at least four, at least five or at least six of the following cytokines IL-10, IL-16, IL-12p40, IFN-γ, IL-6 and TNF-α. In one embodiment, said diagnosing is achieved using the determined abundance of least one, at least two, at least three, at least four, at least five, or at least six of the following cytokines: IL-10, IL-16, IL-12p40, IL-17A, TNF-α, IL-10.

In another embodiment, said diagnosing is achieved using the determined abundance of least one, at least two, or preferably at least three of the following cytokines IL-10, IL-16, IL12p40.

In a preferred embodiment, said diagnosing is achieved using the determined abundance of least IL-10, preferably at least IL-10 and IL-16.

In another embodiment, said diagnosing is achieved using the determined abundance of least one, at least two, or preferably at least three of the following cytokines IL-17A, TNF-α, IL-10.

In a preferred embodiment, said diagnosing is achieved using the determined abundance of least IL-17A, preferably at least IL-17A and TNF-α.

In another embodiment, said diagnosing is achieved using at least the determined abundance of IL-10 and TNF-α.

In another embodiment, said diagnosing is achieved using at least the determined abundance of IL-10 and IL-17A.

In a preferred embodiment, said diagnosis is achieved by further determining the abundance of IL-8.

IL-17A for example, is a cytokine, that is produced by a subpopulation of CD4+ T cells called Th17 cells. Th17 cells are constitutively present in a part of the gut, the lamina propria of the small intestines, due to a specific population of bacteria present there (segmented filamentous bacteria), where they ensure immune surveillance and proper gut function and are quasi absent in other organs such as lung or liver. Infections or other conditions can increase the Th17 cell population, and once activated Th17 cells promote the eradication of extracellular bacteria, and fungi such as Candida albicans. TNF-α is a pro-inflammatory cytokine that is produced by several cell types including T lymphocytes, macrophages, neutrophils, astrocytes, glia cells, fibroblasts, and smooth muscle cells in response to injury and inflammatory stimuli. IL-10 is a cytokine with anti-inflammatory properties which has a central role in preventing inflammatory and autoimmune pathologies. While it was originally demonstrated to be produced by CD4+T helper type 2 (Th2) cells, many immune cell types could produce it, including Th1 and regulatory T cells, CD8+ T cells, B cells, macrophages, dendritic cells, neutrophils and eosinophils. Notably, some nonhematopoietic cell types, such as epithelial cells, can also produce IL-10. IL-10 production in the brain has also been described, but the cellular sources remained to be identified. Several preclinical and clinical studies have suggested a possible role of IL-10 in brain function and behavior.

The abundance of the biomarkers in the blood sample is for example the concentration of said biomarkers in the blood sample, in the serum part of said blood sample or in the plasma part of said blood sample. For measuring the abundance of the plurality of the biomarkers in the collected sample of blood according to the invention, various methods, that are well-known from the man skilled in the art, may be used. For example, these measures may be carried out using electro-chemo-luminescence (ECL)-based or bead-based immunoassays.

Preferably, the diagnosing step is a differential diagnosing of the bipolar disorder and the major depressive disorder as above, but excluding the following cytokines CCL2, CCL3, CCL4, CCL11, CCL13, CCL17, CCL20, CCL22, CCL26, CXCL10, IL-1-α, IL-1-β, IL-2, IL-4, IL-5, IL-7, IL-8, IL-12p70, IL-13, IL-15, IL-21, IL-22, IL-23, IL-31 and TNF-β.

For the implementation of the invention, a probability p that a patient is bipolar instead of suffering from a MDD can be determined. It is to be noted that, as the diagnosing is a differential diagnosing, the probability that a patient is bipolar is a probability that a patient is bipolar instead of suffering of a major depressive disorder. It is also noted determining a probability that a patient is bipolar or determining a probability that a patient is having MDD is the same in the context of the invention wherein a differential diagnosing is achieved.

For example, the implementation of the method according to the invention could be done by replacing the variables “A”, “B”, “C”, “D”, “E”, “F”, “G” and “H” in equation below by the appropriate values as described:

$$p = \frac{1}{1 + {e}^{{β}_{0} + {β}_{1} A + {β}_{2} B + {β}_{3} C + {β}_{4} D + {β}_{5} E + {β}_{6} F + {β}_{7} G + {β}_{8} H}}$$

wherein:
the variable “A” is equal to 1 if the patient has been treated with benzodiazepine, and equal to 0 in the opposite case; variable “B” is equal to 1 if the patient has been treated with antidepressants, and equal to 0 in the opposite case; variable “C” is equal to 1 if the patient has been treated with neuroleptics or atypical antipsychotics, and equal to 0 in the opposite case; variable “D” is equal to 1 if the patient has been treated with any other antipsychotics, and equal to 0 in the opposite case; variable “E” is equal to 1 if the patient has been treated with lithium, and equal to 0 in the opposite case;
variable “F” is equal to the serum concentration expressed in pg/ml of a first cytokine among IL-17A, TNF-α, IL-10, IL-16, IL-12p40, IFN-γ, IL-15 and IL-27, for example IL-17A; variable “G” is equal to the serum concentration of a second cytokine expressed in pg/ml among IL-17A, TNF-α, IL-10, IL-16, IL-12p40, IFN-γ, IL-15 and IL-27, for example TNF-α; and variable “H” is equal to the serum concentration expressed in pg/ml of a third cytokine among IL-17A, TNF-α, IL-10, IL-16, IL-12p40, IFN-γ, IL-15 and IL-27, for example IL-10; and
β₀ is comprised between −0.895 and −0.733 preferentially equal to approximately −0.814, more preferentially equal to −0.814; β₁ is comprised between 0.552 and 0.674 preferentially equal to approximately 0.613, more preferentially equal to 0.613; β₂ is comprised between −0.541 and −0.443 preferentially equal to approximately −0.492, more preferentially equal to −0.492; β₃ is comprised between 0.262 and 0.320 preferentially equal to approximately 0.291, more preferentially equal to 0.291; β₄ is comprised between 0.304 and 0.372 preferentially equal to approximately 0.338, more preferentially equal to 0.338; β₅ is comprised between 0.231 and 0.283 preferentially equal to approximately 0.257, more preferentially equal to 0.257; β₆ is comprised between 0.281 and 0.343 preferentially equal to approximately 0.312 if the cytokine is IL17A, more preferentially equal to 0.312; β₇ is comprised between 0.112 and 0.136, preferentially equal to approximately 0.124 if the cytokine is TNF-α, more preferentially equal to 0.124; and β₈ is comprised between 0.171 and 0.209 preferentially equal to approximately 0.190, more preferentially equal to 0.190 if the cytokine is IL-10.

The probability p above is then advantageously calculated taking into account that the patient has been treated using benzodiazepine, antidepressants, neuroleptics or atypical antipsychotics, and/or any other antipsychotics, or not. If the calculation of the probability does not take into account treatments of the patient using the above drugs, then the corresponding coefficient β₁, β₂, . . . , β₅ are considered as equal to zero. The probability p is finally calculated on the basis of at least one, at least two or at least three cytokines among IL-17A, TNF-α, IL-10, IL-16, IL-12p40, IFN-γ, IL-15 and IL-27, for example IL-17A, TNF-α and IL-10, depending of the values of β₆, β₇ and β₈. Additional cytokines, for example IL-6 and IL-8 may form additional members of the equation, for example to improve the accuracy of the probability p that is calculated. They will be calculated taking into account additional coefficients β₉, β₁₀, β₁₁ and β₁₂, respectively, and their respective serum concentration I, J, K and L expressed in pg/ml.

Hence, the probability p is calculated using an equation that is of the type:

$$p = \frac{1}{1 + {e}^{X}}$$

wherein X comprises one or an addition of two or more terms, including at least one of the following terms β₆F, β₇G, and β₈H.

Thus, according to the invention, at least one, at least two or at least three cytokines among TNF-α, IFN-γ, IL-6, IL-10, IL-12p40, IL-15, IL-16, IL-17A and IL-27, for example IL-17A, TNF-α and IL-10, or, in another example, TNF-α and/or IL-10 are used as biomarkers to achieve a differential diagnosis of bipolar disorders and major depressive disorders. Indeed, those cytokines have been identified that, when combined, discriminate bipolar and unipolar patients for example after adjustment to past or ongoing treatments with antidepressants, benzodiazepines, antipsychotics or lithium. While these three cytokines have already been associated with affective disorders in humans and behavioral alterations in mice, this study is the first one to demonstrate that they can be used to discriminate BD and MDD patients. The invention pave the way for the development of a blood-based assisted clinical decision support system for the differential diagnosis of bipolar disorder and major depressive disorder patients.

### Example 1

1. Methods and Material

Eligible study participants were 148 adults diagnosed with current Major Depressive Episode (MDE) and 100 age- and gender-matched healthy controls from a study registered in ClinicalTrials.gov with ID: NCT02209142. Participants had a clinical evaluation using the Semi-Structured Clinical Interview of the 4th edition of the Diagnostic and Statistical Manual of Mental Disorders (DSM). Patients were recently admitted in a psychiatric unit or have been recently referred to a psychiatrist for a MDE. The diagnosis of MDE was made by skilled psychiatrists based on the DSM-IV criteria of mood disorders and the MDE severity was evaluated by the 17-item Hamilton Depression Rating Scale (HDRS). Patients were included if they scored 19 or higher on the HDRS. Exclusion criteria were a history of substance use disorder in the past 12 months, a diagnosis of schizophrenia, psychotic or schizoaffective disorder according to the DSM-IV, a severe progressive medical disease, pregnancy, vaccination within a month before the inclusion in the study and being under 18. Fourteen patients were excluded due to the manifestation of exclusion criteria during the study (diagnosis of severe medical conditions, consent withdrawal) or unavailable gene expression data and/or main outcome measures. Finally, data from 134 MDE patients were included in the analyses. Former and ongoing patient treatments were recorded including those involving the use of selective serotonin reuptake inhibitors (SSRIs), tricyclic antidepressants (TCAs), monoamine oxidase inhibitors (MAOIs), benzodiazepines, atypical antipsychotics, lithium, sysmotherapy, transcranial magnetic stimulation (TMS), structured psychotherapy, valproate salts, carbamazepine and lamotrigine. All participants received a full explanation of the procedure and signed a written consent form before their participation.

Peripheral blood samples were obtained from fasting subjects between 7:00 am and 9:00 am on workdays. Five milliliters of peripheral blood were drawn by venipuncture into serum Vacutainer tubes. For the serum collection, the blood was allowed to clot for 1 h before centrifugation (1500×g, 10 min). The serum and plasma samples were stored in 0.5 ml aliquots at −80° C. For the measurements of cytokine and antibody levels, serum samples were thawed on ice, and 50 μl aliquots were prepared and stored at −80° C.

Serum levels of CC chemokine ligand (CCL)2, CCL3, CCL4, CCL11, CCL13, CCL17, CCL20, CCL22, CCL26, CXC chemokine ligand (CXCL)10, IL-1-a, IL-1-β, IL-2, IL-4, IL-5, IL-6, IL-7, IL-8, IL-10, IL-12p40, IL-12p70, IL-13, IL-15, IL-16, IL-17A, IL-21, IL-22, IL-23, IL-27, IL-31, interferon (IFN)-γ, Tumor Necrosis Factor (TNF)-α, TNF-β, Granulocyte-Macrophage Colony-Stimulating Factor (GM-CSF), Vascular Injury Growth Factor (VEGF)-A, soluble intercellular adhesion molecule (sICAM)-1 and soluble vascular endothelial cell adhesion molecule (sVCAM)-1, C Reactive Protein (CRP), serum amyloide A protein (SAA), S100B and Glial fibrillary acidic protein (GFAP) were measured using the Proinflammatory Panel 1, Cytokine Panel 1, Chemokine Panel 1 Vascular Injury Panel II V-PLEX™ kits (MesoScale Discovery (MSD)). All assays were performed according to the manufacturer's instructions. The data were acquired on the V-PLEXR Sector Imager 2400™ plate reader and analyzed using the Discovery Workbench™ 3.0 software (MSD). The standard curves for each cytokine were generated using the premixed lyophilized standards provided in the kits. Serial 2-fold dilutions of the standards were run to generate a 13-standard concentration set, and the diluent alone was used as a blank. The cytokine concentrations were determined from the standard curve using a 4-parameter logistic curve fit to transform the mean light intensities into concentrations. The Lower Limit Of Detection (LLOD) was a calculated concentration corresponding to the average signal 2.5 standard deviations above the background (zero calibrator).

In univariate analysis, Student's t-test and Mann-Whitney-Wilcoxon test were performed to assess statistical significance of Gaussian and non-Gaussian distributed data respectively. To develop a predictive model for remission we used the elastic net, which is a regularized regression model, i.e. generalized linear model with penalties to avoid extreme parameters that could cause overfitting. Elastic net is also a method of selection of variables that addresses the issue of multicollinearity that arises in the dataset because cytokines and chemokines are not independent of each other. To minimize variation across testing datasets, we repeated 5-fold cross-validation 200 times with independent random dataset partitions to optimize stability. The hyper-parameters alpha and lambda were tuned 10 times for each partition via 5-fold cross-validation with the optimal tuning parameter values chosen to maximize the area under the Receiver Operating Characteristics (ROC) curve (AUC). Weighted mean coefficient values (β) were calculated using the proportion of drawings in which the coefficient was different from zero (meaning the associated variable was selected as important) as the ponderation. All statistical analyses were performed using the R software packages Stats™, Caret™, Glmnet™, pROC™, eNetXplorer™.

2. Results

2.1 Serum Protein Levels in MDD and BD Patients

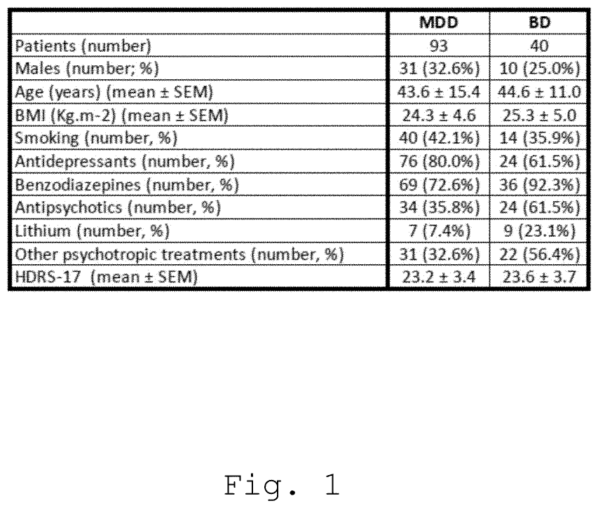

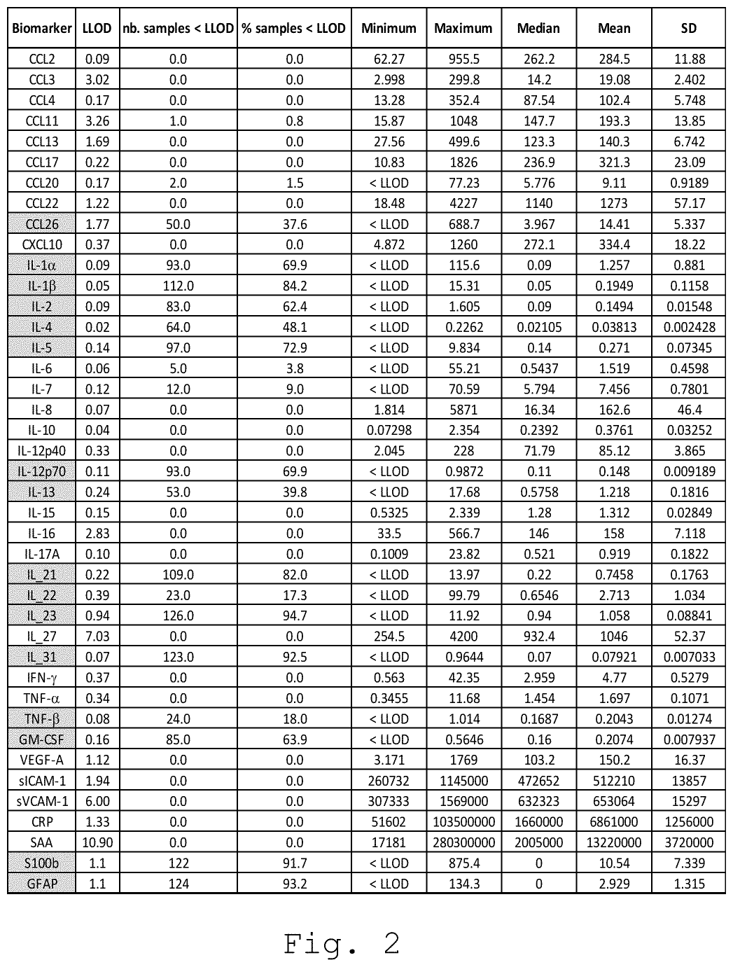

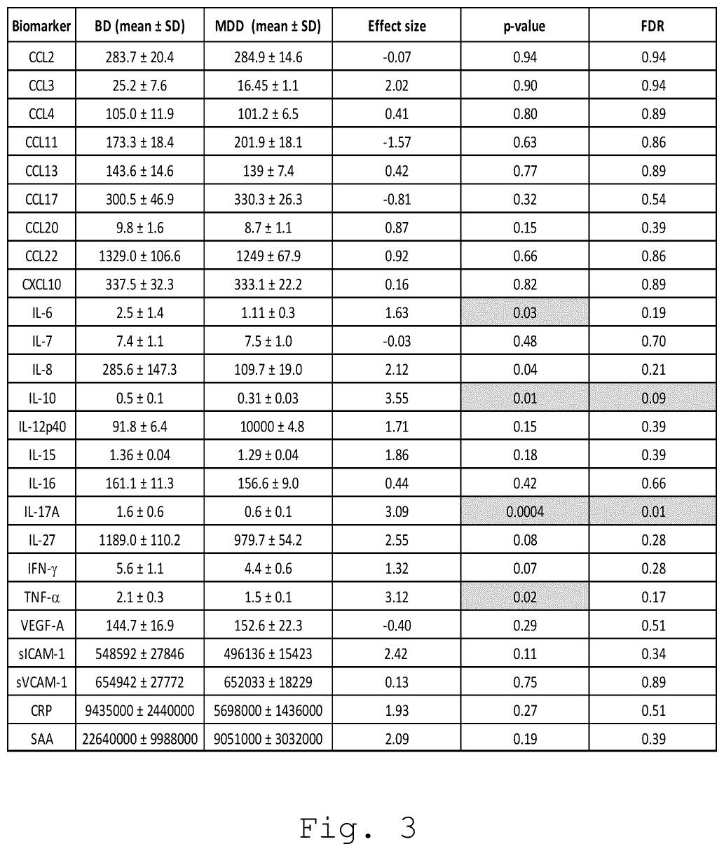

As shown in FIG. 1, the MDE patients were extensively characterized for clinical features including age, body-mass-index (BMI), tobacco consumption, past or on-going treatments with antidepressants, antipsychotics, benzodiazepine and lithium. They were also assessed for depression-associated symptoms using the Hamilton Depression Rating Scale (HDRS). The serum samples were analysed for 12 chemokines (CCL2, CCL3, CCL4, CCL11, CCL13, CCL17, CCL20, CCL22, CCL26, CXCL10), 15 interleukins (IL-1-α, IL-1-β, IL-2, IL-4, IL-5, IL-6, IL-7, IL-8, IL-10, IL-12p40, IL-12p70, IL-13, IL-15, IL-16, IL-17A, IL-21, IL-22, IL-23, IL-27, IL-31), three inflammatory cytokines (IFN-γ, TNF-α, TNF-β, two growth factors (GM-CSF and VEGFA), two proteins produced by liver in response to inflammation (CRP, SAA), two biomarkers of vascular injury (sICAM-1 and sVCAM-1) and two biomarkers of brain-blood barrier (BBB) permeability. As shown in FIG. 2, among the 41 analyzed proteins, 16 were below the LLOD in more than 10% of samples and were not included in downstream analyses. In an exploratory analysis, the levels of the 35 remaining proteins were compared in BD and MDD patients using univariate analysis. After correction for multiple testing, and as shown in FIG. 3, it was found that both IL-17A and IL-10 were expressed at higher levels in BD patients compared to MDD patients. To a lesser extent, it is the case as well for IL-6, IL-8, IL-27, IFN-γ.

2.2 Multivariate Classifications

Most cytokines and chemokines belong to common biochemical or functional pathways. Furthermore, their production could be impacted by patient clinical characteristics including age, gender, body mass index (BMI) and tobacco consumption. Most importantly, several studies have suggested that some psychotropic drugs including antidepressants, antipsychotics and lithium may impact serum levels of cytokines and chemokines and other inflammatory biomarkers. In contrast to univariate statistical methods that assess the differential expression of individual proteins without considering the relationships between them or with other variables, classification methods are used to establish a prediction model based on samples with known class outcomes (e.g. BD or MDD). A set of biomarkers with the best joint discriminatory ability to differentiate between the classes is identified (predictor selection), and the resulting prediction model is used to predict the class outcomes of new patient samples. Unlike univariate methods, these multivariable methods consider the relationships between candidate biomarker proteins, and seek to capture the differences between the sample groups on a multi-feature level.

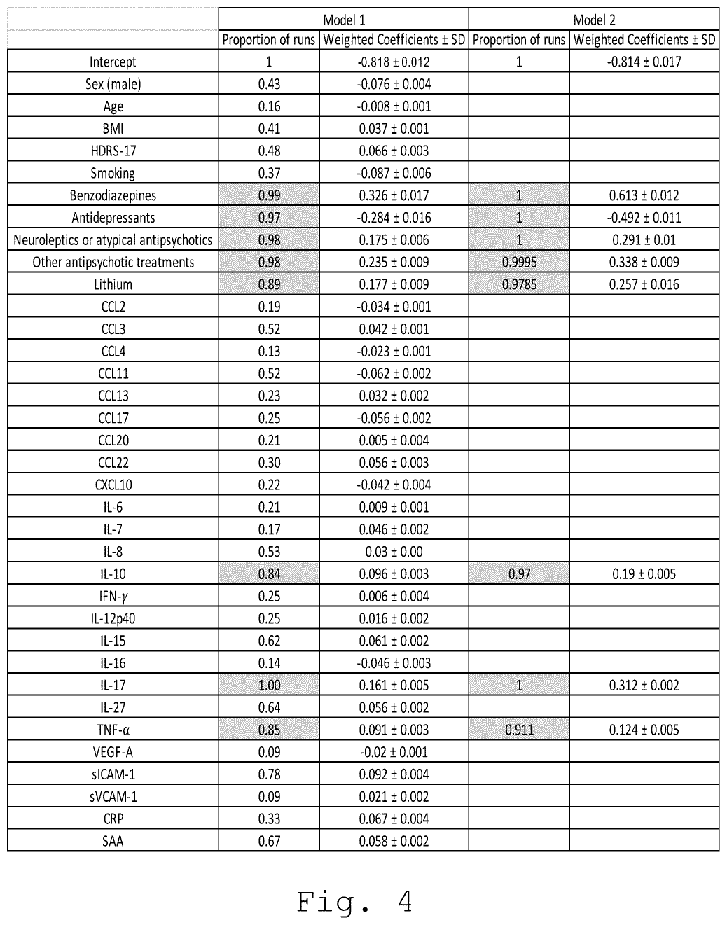

One of these multivariate methods, termed regularized logistic regression, was used to identify serum biomarkers that could discriminate BP and MDD patients after adjustment for covariates known or suspected to either impact serum biomarker levels and/or being associated with one of the two diagnosis categories: gender, age, BMI, tobacco consumption, and former or ongoing treatments with antidepressants, antipsychotics, benzodiazepines and lithium and HDRS-17 score. IL-17A was selected as a determinant feature in 1997 runs out of 2000 (FIG. 4). TNF-α and IL-10 were also selected in more that 80% of drawings, i.e. 1849 and 1836 respectively (FIG. 4). High serum levels of these three cytokines were all associated with decreased odds of belonging to the BD diagnosis category and the predictive value of this model was good (mean AUC=0.698±0.098; mean sensitivity: 95.51±2.05%; mean specificity: 18.99±7.55%).

Ideally, a blood-based diagnosis algorithm should not only be specific and sensitive, but also relying on a small number of biomarkers. As IL-17A, IL-10 and TNF-α were those that were selected the most frequently in random training/test drawings, we further estimated the predictive value of these three combined cytokines after adjustment to medications which were selected as determinant features in Model 1. The predictive value of this new model was higher (mean AUC=0.80±0.07), and its sensitivity and specificity were 94.70±0.01% and 35.17±0.05% respectively. Altogether, the data show that serum levels of IL-17A and/or IL-10 and/or TNF-α, preferably these three cytokines in combination, can serve as diagnosis biomarkers to differentiate MDD and BD patients.

It is to be noted that the Model 2 as appearing in FIG. 4 could be used to determine the probability (p) that a patient is bipolar.

This could be done by replacing the variables “A”, “B”, “C”, “D”, “E”, “F”, “G” and “H” in equation below by the appropriate values as described:

$$p = \frac{1}{1 + {e}^{- 0.814 + 0.613 A + - 0.492 B + 0.291 C + 0.338 D + 0.257 E + 0.312 F + 0.124 G + 0.190 H}}$$

wherein:

the variable “A” is equal to 1 if the patient has been treated with benzodiazepine, and equal to 0 in the opposite case; variable “B” is equal to 1 if the patient has been treated with antidepressants, and equal to 0 in the opposite case; variable “C” is equal to 1 if the patient has been treated with neuroleptics or atypical antipsychotics, and equal to 0 in the opposite case; variable “D” is equal to 1 if the patient has been treated with any other antipsychotics, and equal to 0 in the opposite case; variable “E” is equal to 1 if the patient has been treated with lithium, and equal to 0 in the opposite case; variable “F” would be equal to IL-17A serum concentration expressed in pg/ml; variable “G” is equal to TNF-α serum concentration expressed in pg/ml; and variable “H” is equal to IL-10 serum concentration expressed in pg/ml.

Another of these multivariate methods was used, namely the Elastic Net framework, which has good performance when the number of events per variable is low, and addresses the issue of multicollinearity while performing variable selection and computing estimates. The more frequently a variable is selected by the Elastic Net, the more likely it is stably associated with the outcome. This frequency, coined Variable Inclusion Probability (VIP), is a measure of the stability of an association in the absence of asymptotically valid p-values which are not available in regularized regression. The VIP can be interpreted as the posterior probability of including a variable in the model. After defining an appropriate threshold, the VIP can be used to select predictors for use in follow-up analyses. However, determining an appropriate threshold for the VIP can be challenging. It has been recommended the use of a “conservative threshold of 50%” because their goal was “not to miss any possibly relevant predictors”. However, this 50% threshold increases the risk of false positives. Hence, VIP was considered above 80% to identify variables stably associated with belonging to the BD diagnosis categories.

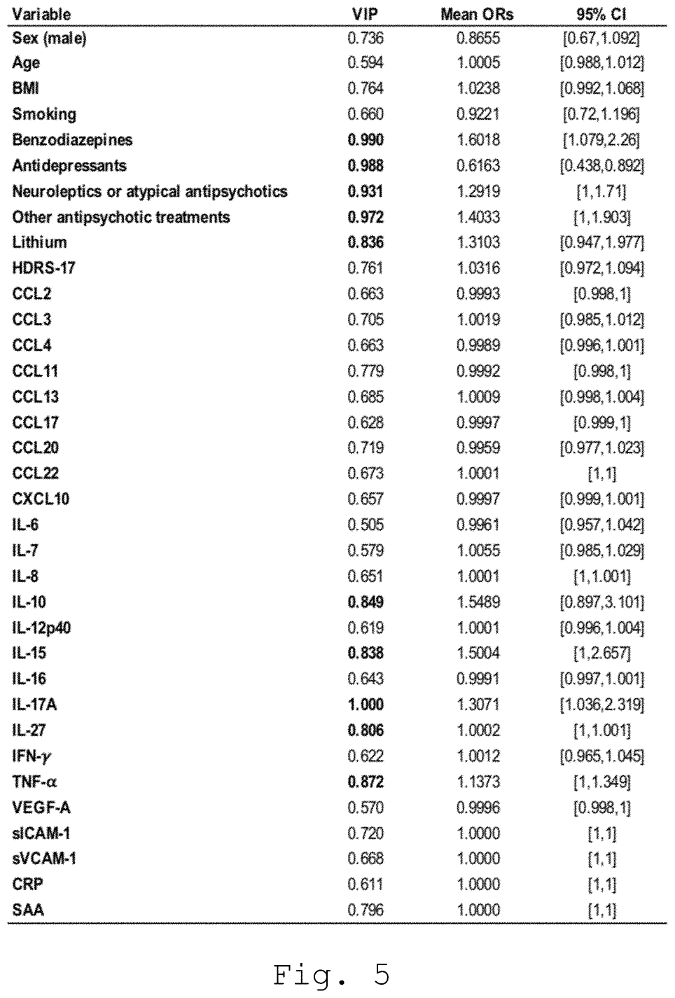

IL-17A was selected as a determinant feature in all (1000 out of 1000) resampled models, as show in FIG. 5. Also, as shown in this Figure, TNF-α, IL-10, IL-15 and IL-27 were also selected in more than 80% of resamples. High serum levels of these biomarkers were all associated with increased odds of belonging to the BD diagnosis category. The mean accuracy of this model was 0.80 (95% CI 0.75-0.84) as determined by the AUROC. The optimal cut-off was then determined for maximizing both sensitivity and specificity. This mean cut-off was 0.33 (95% CI 0.21-0.47) and it resulted in a sensitivity and specificity of 0.71 (95% CI 0.55-0.88) and 0.82 (95% CI 0.60-0.95) respectively. This model had a positive predictive value of 0.64 (95% CI 0.49-0.82) and a negative predictive value of 0.89 (95% CI 0.82-0.92). To visualize the prediction capability of the resampled models, as shown in FIG. 5, the distribution of predicted probabilities for individuals to be classified as belonging to the BD category was plotted. The distribution of probabilities for individuals clinically diagnosed with MDD was skewed towards lower values (peak at 0.2) meaning that the model predicted a low probability to belong to the BD category. The distribution of probabilities for individuals clinically diagnosed with BD was more uniform in agreement with a low-to-moderate sensitivity, even with a cut-off of 0.33 which maximized both sensitivity and specificity.

### Example 2

1. Methods and Material

1.1 Study Subjects

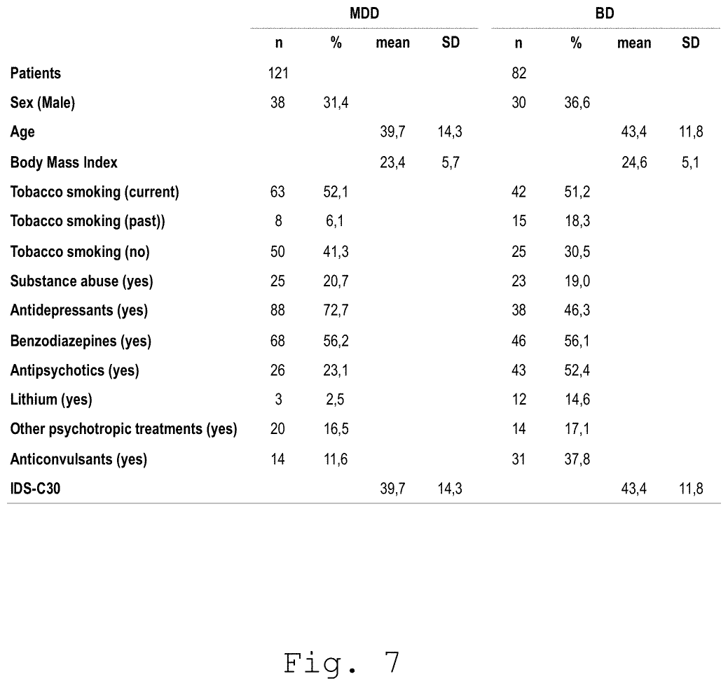

A total of 203 adults diagnosed with current MDE were recruited under informed consent. FIG. 7 details the patients clinical characteristics that were considered in this cohort. Participants had a clinical evaluation using the Semi-Structured Clinical Interview of the 4th edition of the Diagnostic and Statistical Manual of Mental Disorders. Patients were recently admitted to a psychiatric unit or had been recently referred to a psychiatrist. The diagnosis of MDE was made by experienced psychiatrists based on the DSM-IV criteria of mood disorders and the MDE severity was evaluated by the IDS-C30. Exclusion criteria were a diagnosis of schizophrenia, psychotic or schizoaffective disorder according to the DSM-IV, a severe progressive medical disease and pregnancy. 203 MDE patients were included in the analyses. Current treatments including use of selective serotonin reuptake inhibitors (SSRIs), tricyclic antidepressants (TCAs), monoamine oxidase inhibitors (MAOIs), benzodiazepines, atypical antipsychotics, lithium, anticonvulsants, electroconvulsive therapy (ECT), valproate salts, carbamazepine and lamotrigine, were recorded. All subjects provided written informed consent after receiving a complete description of the study.

1.2 Blood Samples

Venous blood was obtained from fasting subjects between 7:00 am and 9:00 am on weekdays. Five milliliters of peripheral blood were drawn by venipuncture and allowed to clot for 1 h before centrifugation (1500×g, 10 min). Serum samples were stored in 0.5 ml aliquots at −80° C. For measurements of cytokines, serum samples were thawed on ice.

1.3 Immunoassays

Serum samples were assessed for levels of C-C motif chemokine ligand IL-1α, IL-1β, IL-2, IL-4, IL-5, IL-6, IL-7, IL-8, IL-10, IL-12p40, IL-12p70, IL-13, IL-15, IL-16, IL-17A, IFN-γ, TNF-α, TNF-β, GM-CSF and VEGF-A. For this purpose, the Proinflammatory Panel 1 and Cytokine Panel 1 V-PLEX® kits were used according to the manufacturer's instructions. Data were acquired on the V-PLEX® Sector Imager 2400 plate reader and analyzed using the Discovery Workbench 3.0 software. Standard curves for each cytokine were generated using the premixed lyophilized standards provided with the kits. Serial 2-fold dilutions of the standards were run to generate a 13-standard concentration set, and the diluent alone was used as a blank. Cytokine concentrations were determined by extrapolation from the standard curve using a 4-paramater logistic curve fit to transform the mean light intensities into concentrations. The LLOD was determined as the lowest concentration of an analyte yielding a signal equal or over 2.5 standard deviations above blank (zero calibrator).

1.4 Statistical Analyses

We used a combined the nonparametric bootstrap with a regularized logistic regression model to assess the influence of individual biomarker on the prediction of MDD/BD. Applying the nonparametric bootstrap method, by constructing 1000 resamples with replacement from the original dataset (and with equal size), produced a bootstrap distribution for each regression coefficient. The information in this bootstrap distribution was used to assess the influence of each predictor in the model and derive confidence intervals for the predictors and measures of performance. We used the Elastic Net as the regularization regression method, because it is able to integrate the issue of multicollinearity that arises from the lack of independence between biomarkers. All statistical analyses were performed using the R software packages Stats, Caret, Glmnet, pROC and rms.

2. Results

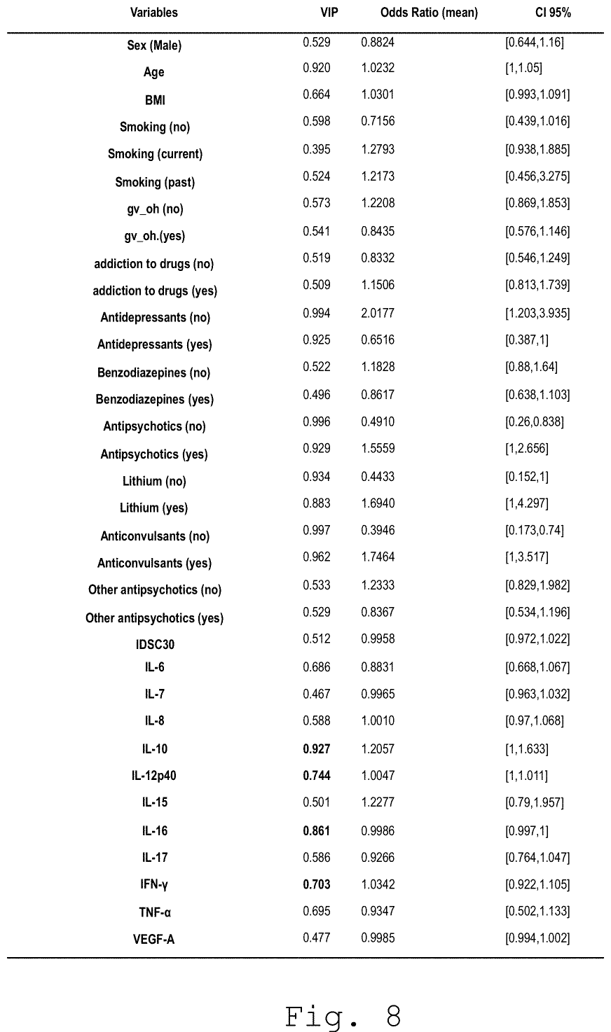

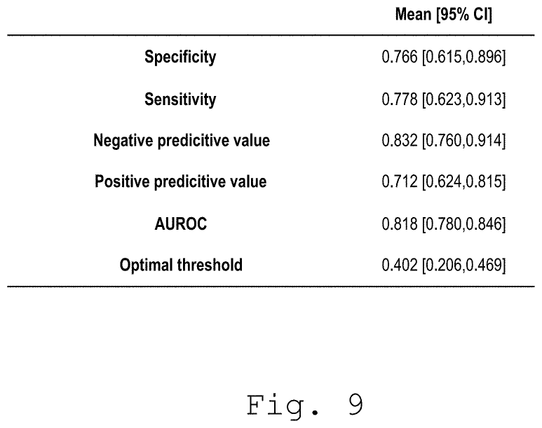

Among the 20 proteins analyzed, 9 were either below the LLOD in more than 10% of samples or within a range for which the inter-assay variability was >5%. This prompted us to retain 25 biomarkers in downstream analyses: IL-6, IL-7, IL-8, IL-10, IL-12p40, IL-15, IL-16, IL-17A, IFN-γ, TNF-α and VEGF-A. We considered VIP above 65% to identify variables stably associated with belonging to the BD diagnosis categories. As shown in FIG. 8, IL-10, IL-16, IL-12p40, IFN-γ, TNF-α and IL-6 were selected as a determinant feature in 92.7%, 86.1%, 74.4%, 70.3%, 69.5% and 68.6% of the resampled models respectively. The mean accuracy of this model was 0.818 (95% CI 0.780,0.846) as determined by the area under the ROC curve (AUROC). This is illustrated in FIG. 9. It was then determined the optimal cut-off for maximizing both sensitivity and specificity. This mean cut-off was 0.402 (95% CI 0.206,0.469) and it resulted in a sensitivity and specificity of 0.778 (95% CI 0.623,0.913) and 0.766 (95% CI 0.616,0.896) respectively. This model had a positive predictive value of 0.712 (95% CI 0.624,0.815) and a negative predictive value of 0.832 (95% CI 0.760,0.914).

1. An in vitro or ex vivo method for differentially diagnosing a bipolar disorder and a major depressive disorder in a human patient in a need thereof presenting depressive symptoms, comprising:
determining, from a biological sample, the abundance of at least one of the following cytokines TNF-α, IFN-γ, IL-6, IL-10, IL-12p40, IL-15, IL-16, IL-17A, IL-27; and
diagnosing a bipolar disorder or a major depressive disorder from the determination of the abundance of the at least one of the following cytokines: TNF-α, IFN-γ, IL-6, IL-10, IL-12p40, IL-15, IL-16, IL-17A, IL-27.

2. The method according to claim 1, comprising:
determining, from the biological sample, the abundance of at least two of the following cytokines: TNF-α, IFN-γ, IL-6, IL-10, IL-12p40, IL-15, IL-16, IL-17A, IL-27; and
diagnosing a bipolar disorder or a major depressive disorder from the determination of the abundance of the at least two of the following cytokines: TNF-α, IFN-γ, IL-6, IL-10, IL-12p40, IL-15, IL-16, IL-17A, IL-27.

3. The method according to claim 1, wherein the diagnosing is achieved using the determined abundance of
at least one of the following cytokines: IL-17A, TNF-α, IL-10, IL-15, IL-27; and
at least one of the following cytokines: IL-10, IL-16, IL-12p40, IFN-γ, IL-6, TNF-α.

4. The method according to claim 1, comprising:
determining, from the biological sample, the abundance of at least one, at least two or at least three of the following cytokines: IL-17A, TNF-α; IL-10; and
diagnosing a bipolar disorder or a major depressive disorder from the determination of the abundance of at least one, at least two of the following cytokines: IL-17A, TNF-α, IL-10.

5. The method according to claim 1, wherein the diagnosis is achieved using at least the determined abundance of at least one of the following cytokines: IL-10, TNF-α.

6. The method according to any claim 1, wherein the diagnosis is achieved using at least the determined abundance of IL-10 and TNF-α.

7. The method according to claim 1, wherein the diagnosis is achieved using the determined abundance of one or more additional cytokines including IL-8.

8. The method according to claim 1, wherein the diagnosing excludes taking into account the abundance of the following cytokines: CCL2, CCL3, CCL4, CCL11, CCL13, CCL17, CCL20, CCL22, CCL26, CXCL10, IL-1-α, IL-1-β, IL-2, IL-4, IL-5, IL-7, IL-8, IL-12p70, IL-13, IL-15, IL-21, IL-22, IL-23, IL-31 and TNF-β into the provided biological sample.

9. The method according to claim 1, wherein the biological sample is selected from the group consisting of blood, biopsy tissue, blood serum, blood plasma, stool, sputum, cerebrospinal fluid, and supernatant from cell lysate.

10. The method according to claim 9, wherein the biological sample is selected from the group consisting of blood, blood plasma, and blood serum.

11. The method according to claim 1, further comprising:
determining, from a biological sample, the abundance of at least one of the following cytokines: TNF-α, IFN-γ, IL-6, IL-10, IL-12p40, IL-15, IL-16, IL-17A, IL-27, and
comparing the abundance level of the at least one cytokines with reference values.

12. The method according to claim 1, further comprising determining a probability p that a patient is bipolar.

13. The method of claim 12, wherein the probability p is calculated using the following equation:
$$p = \frac{1}{1 + {e}^{X}}$$
wherein X comprises one or an addition of two or more terms βᵢSRi, wherein the variable SRi is equal to the serum concentration of one of the cytokines TNF-α, IFN-γ, IL-6, IL-10, IL-12p40, IL-15, IL-16, IL-17A and IL-27, and value βᵢ is a reference value identified for said cytokine.

14. The method of claim 12, wherein the probability p is calculated using the following equation:
$$p = \frac{1}{1 + {e}^{X}}$$
wherein X comprises one or an addition of two or more terms, including at least one of the following terms β₆F, β₇G, and β₈H, wherein the variable “F” is equal to IL-17A serum concentration; variable “G” is equal to TNF-α serum concentration; and variable “H” is equal to IL-10 serum concentration.

15. The method of claim 14, wherein the serum concentration of at least one cytokine selected from the group consisting of IL-17A, TNF-α and IL-10 are expressed in pg/ml.

16. The method according to claim 14, wherein
β₆ is comprised in a range of from 0.281 to 0.343;
β₇ is comprised in a range of from 0.112 to 0.136; and
β₈ is comprised in a range of from 0.171 to 0.209.

17. The method according claim 12, wherein the probability is calculated taking into account whether the patient has been treated using one or more of the following: benzodiazepine, antidepressants, neuroleptics or atypical antipsychotics, other antipsychotics.

18. Method of performing differential diagnosis of a bipolar disorder or a major depressive disorder, comprising relying on at least one, at least two or at least three of the following cytokines: TNF-α, IFN-γ, IL-6, IL-10, IL-12p40, IL-15, IL-16, IL-17A, IL-27 as biomarkers.

19. The method according to claim 1, comprising:
determining, from the biological sample, the abundance of at least three of the following cytokines: TNF-α, IFN-γ, IL-6, IL-10, IL-12p40, IL-15, IL-16, IL-17A, IL-27; and
diagnosing a bipolar disorder or a major depressive disorder from the determination of the abundance of the at least three of the following cytokines: TNF-α, IFN-γ, IL-6, IL-10, IL-12p40, IL-15, IL-16, IL-17A, IL-27.

20. The method according to claim 1, wherein the diagnosing is achieved using the determined abundance of
at least two of the following cytokines: IL-17A, TNF-α, IL-10, IL-15, IL-27; and
at least two of the following cytokines: IL-10, IL-16, IL-12p40, IFN-γ, IL-6, TNF-α.
